(19) United States
(12) Patent Application Publication
(10) Pub. No.: US 2020/0163559 A1
(43) Pub. Date: May 28, 2020

(54) HEALTH MONITORING DEVICE

(71) Applicant: Microjet Technology Co., Ltd., Hsinchu (TW)

(72) Inventors: Hao-Jan Mou, Hsinchu (TW); Ching-Sung Lin, Hsinchu (TW); Chih-Kai Chen, Hsinchu (TW); Chi-Feng Huang, Hsinchu (TW); Yung-Lung Han, Hsinchu (TW); Wei-Ming Lee, Hsinchu (TW)

(73) Assignee: Microjet Technology Co., Ltd., Hsinchu (TW)

(21) Appl. No.: 16/689,488

(22) Filed: Nov. 20, 2019

(30) Foreign Application Priority Data
Nov. 22, 2018 (TW) 107141751

(51) Int. Cl. A61B 5/021; A61B 5/00; A61B 5/0225

(52) U.S. Cl. CPC A61B 5/02141; A61B 5/0402; A61B 5/02255; A61B 5/681

(57) ABSTRACT

A health monitoring device is provided. The health monitoring device includes a wearable component, an optical monitoring module and an air-bag positioning assembly. The wearable component is worn on a body part of a subject and is contacted with a skin tissue. The optical monitoring module is disposed inside the wearable component and includes a driving controller, an optical sensor and at least one light-emitting element. A light source emitted by the light-emitting element irradiates on the skin tissue. The optical sensor receives a reflection light and generates a sensing signal accordingly. The driving controller converts the sensing signal into health data information and outputs the health data information. By the air-bag positioning assembly, the wearable component is securely worn on the body part of the subject, and the optical sensor is attached on the skin tissue of the subject for accurately monitoring the health data information.

### FIELD OF THE INVENTION

The present disclosure relates to a health monitoring device, and more particularly to a health monitoring device being securely attached on a skin tissue by an air-bag positioning assembly for performing health measurement.

### BACKGROUND OF THE INVENTION

Nowadays, the pursuit of efficiency and the personal pressure are growing and the awareness of the pursuit of personal health is gradually developing. Thus, the ordinary people will want to regularly monitor or examine their own health conditions. In general, the conventional data measurement of human physiological health information is mainly obtained through a fixed sphygmomanometer or a large-scale detection instrument, which usually includes components such as a motor-driven gas pump, an airbag, a sensor and a gas-releasing valve and a battery. The motor-driven gas pump is prone to generate the frictional loss, and the assembled components thereof are bulky. It is not conducive to regular use. Moreover, if a miniature-sized motor-driven gas pump is used, the frictional loss will be increased and more energy will be consumed.

In order to facilitate the ordinary people to regularly monitor their own health conditions and allow the monitoring device to be carried easily, more and more wearable health monitoring devices are introduced into the market. In view of the common health monitoring devices on the market, they are used for measuring the health conditions by an optical detection method. However, the precision of the optical detection method is not high enough so that the measured data is usually not reliable. If the commercially-available sphygmomanometers or other measuring instruments with higher reliability are used, the instruments have bulky volume and fail to meet the requirements of light weightiness, thinning and easy portability. Usually, in the optical detection method, an optical sensor is disposed on the wearable device, and the wearable device is worn on body parts (e.g., wrist or ankle) for monitoring. The major reason of causing the low precision is that the optical sensor cannot be completely attached on the skin of the subject. Accordingly, an error value is generated, and reliable data about the physiological health of the subject cannot be obtained.

Therefore, there is a need of providing a health monitoring device to address the above-mentioned issues. The health monitoring device is small-sized, miniaturized, portable, power-saving, high-precise and can be customized as a personal health monitoring device.

### SUMMARY OF THE INVENTION

An object of the present disclosure provides a health monitoring device. An air-bag positioning assembly, which includes a gas-collecting actuator and an elastic air bag, is configured for securely disposing and positioning. The gas-collecting actuator transports the gas to the interior of the air bag. Therefore, the wearable component is securely disposed on body part of the subject, and the optical sensor is attached on the skin tissue of the subject for accurately monitoring the health information.

In accordance with an aspect of the present disclosure, a health monitoring device is provided. The health monitoring device includes a wearable component, an optical monitoring module and an air-bag positioning assembly. The wearable component is worn on a body part of a subject and is contacted with a skin tissue. The wearable component has a main body, and the main body has a monitoring opening. The optical monitoring module is disposed inside the main body of the wearable component and includes a driving controller, an optical sensor and at least one light-emitting element. The optical sensor and the light-emitting element are disposed corresponding in position to the monitoring opening, a light source emitted by the light-emitting element irradiates on the skin tissue. The optical sensor receives a reflection light and generates a sensing signal accordingly. The driving controller converts the sensing signal into health data information and outputs the health data information. The air-bag positioning assembly includes a gas-collecting actuator and an elastic air bag. The gas-collecting actuator is disposed inside the main body of the wearable component, and the elastic air bag is disposed on the wearable component. The gas-collecting actuator transports the gas to the interior of the elastic air bag, and the elastic air bag is inflated and elastically protrudes out of the wearable component. The wearable component is securely worn on the body part of the subject, and the optical sensor is attached on the skin tissue of the subject for accurately monitoring the health data information.

The above contents of the present disclosure will become more readily apparent to those ordinarily skilled in the art after reviewing the following detailed description and accompanying drawings, in which:

### BRIEF DESCRIPTION OF THE DRAWINGS

FIG. 1 is a schematic view illustrating a health monitoring device according to an embodiment of the present disclosure applied to an earphone;

FIG. 2A is a schematic cross-sectional view illustrating the health monitoring device of FIG. 1;

FIG. 2B schematically shows the air-bag positioning assembly of the health monitoring device of FIG. 2A inflating the air bag;

FIG. 3 is a schematic view illustrating a health monitoring device according to another embodiment of the present disclosure applied to a wearable ring;

FIG. 4 is a schematic cross-sectional view illustrating the health monitoring device of FIG. 3;

FIG. 5 schematically shows the disposed position of the optical monitoring module of the health monitoring device of FIG. 3;

FIG. 6 schematically shows the air bag of the health monitoring device of FIG. 3 being inflated;

FIG. 7A is schematic cross-sectional view illustrating the gas-collecting actuator of the health monitoring device of the present disclosure;

FIG. 7B to FIG. 7C are cross sectional views illustrating the gas-collecting actuator of FIG. 7A performed in an inflating operation;

FIG. 7D is a cross sectional views illustrating the gas-collecting actuator of FIG. 7A performed in a pressure-releasing operation;

FIG. 8A is a schematic exploded view illustrating the micro pump of the wearable health monitoring device according to the embodiment of the present disclosure;

FIG. 8B is a schematic exploded view illustrating the micro pump of the wearable health monitoring device according to the embodiment of the present disclosure and taken along another viewpoint;

FIG. 9A is a schematic cross-sectional view illustrating the micro pump according to the embodiment of the present disclosure;

FIG. 9B is a schematic cross-sectional view illustrating the micro pump according to another embodiment of the present disclosure;

FIGS. 9C to 9E schematically show the actions of the micro pump of FIG. 9A;

FIG. 10 schematically shows the health monitoring device of the present disclosure worn on a human wrist;

FIG. 11 schematically shows the optical sensor of the health monitoring device of the present disclosure attached on the skin tissue;

FIG. 12 is a schematic exploded view illustrating a micro box pump of the health monitoring device of the present disclosure; and

FIGS. 13A to 13C schematically show the actions of the micro box pump of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this invention are presented herein for purpose of illustration and description only. It is not intended to be exhaustive or to be limited to the precise form disclosed.

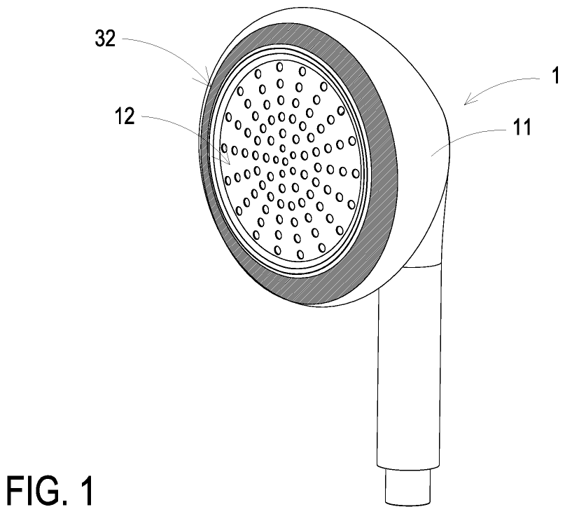

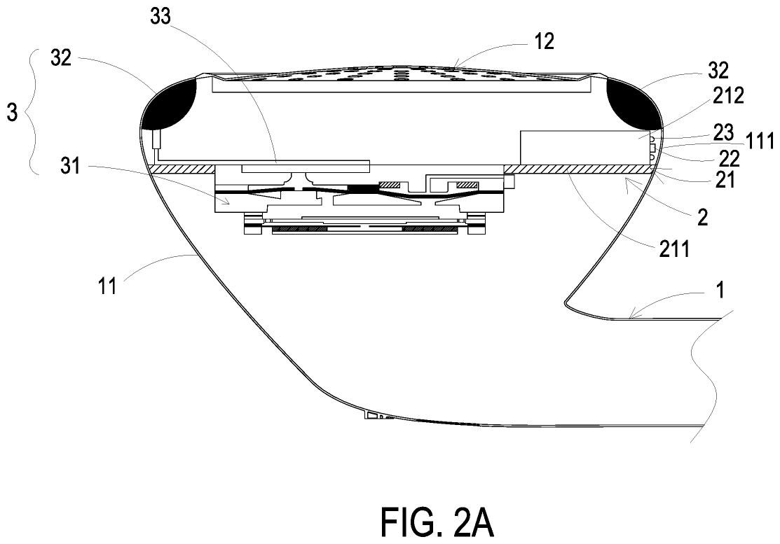

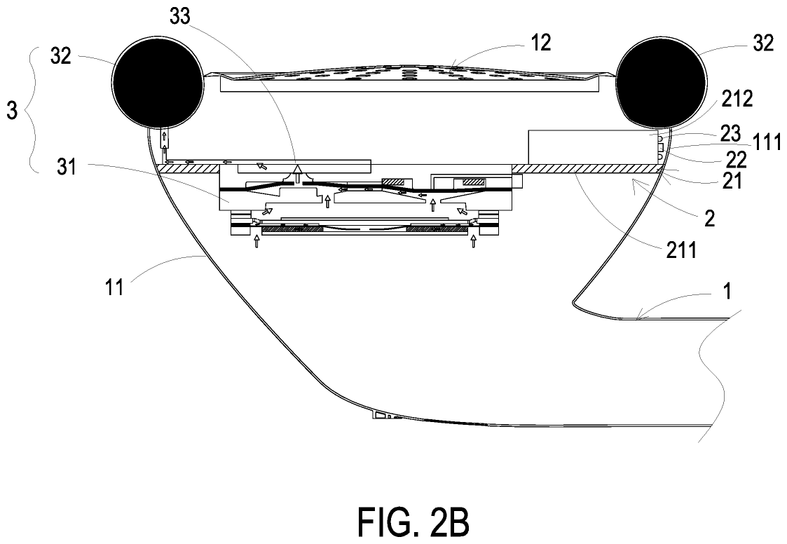

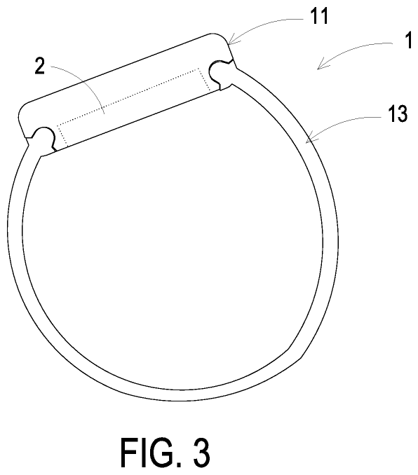

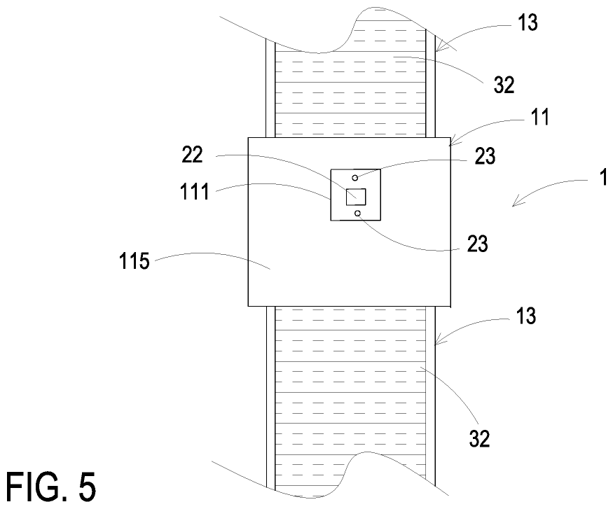

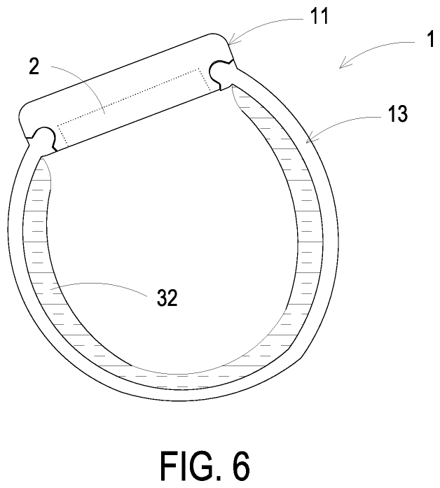

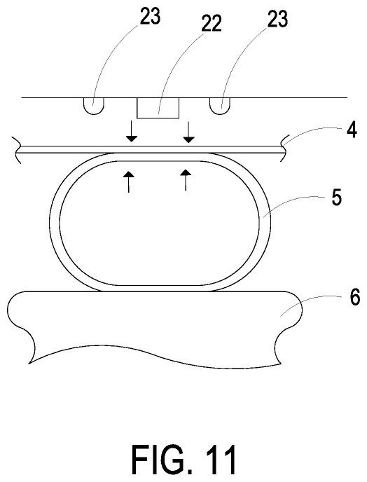

Please refer to FIG. 1, FIG. 2A, FIG. 2B, FIGS. 3 to 6 and FIG. 11. The health monitoring device can be worn on a subject for health monitoring. The health monitoring device includes a wearable component 1, an optical monitoring module 2 and an air-bag positioning assembly 3. The wearable component 1 is worn on the body part of the subject so as to contact the skin tissue 4. The wearable component 1 may be a headphone or wearable ring (e.g., bracelet or watch), but not limited thereto. In an embodiment, the wearable component 1 has a main body 11, and the main body 11 has a monitoring opening 111. The optical monitoring module 2 is disposed inside the main body 11 of the wearable component 1 and includes a driving controller 21, an optical sensor 22 and at least one light-emitting element 23. The optical sensor 22 and the light-emitting element 23 are corresponding in position to the monitoring opening 111 of the main body 11. Therefore, the light source emitted by the light-emitting element 23 irradiates on the skin tissue 4 of the subject, and the optical sensor 22 receives the reflection light and generates a sensing signal accordingly. The driving controller 21 converts the sensing signal into health data information and outputs the health data information. The air-bag positioning assembly 3 includes a gas-collecting actuator 31 and an elastic air bag 32. The gas-collecting actuator 31 is disposed inside the main body 11 of the wearable component 1, and the elastic air bag 32 is disposed on the wearable component 1. The gas-collecting actuator 31 transports the gas inside the wearable component 1, and the elastic air bag 32 is inflated and elastically protrudes out of the the wearable component 1 (as shown in FIG. 2B and FIG. 6). Therefore, the wearable component 1 can be securely worn on the body part of the subject, and allows the optical sensor 22 to be attached on the skin tissue 4 of the subject (as shown in FIG. 11) and to accurately monitor the health data information. The health data information may include a heart rate data, an electrocardiogram data and a blood pressure data.

Please refer to FIG. 1, FIG. 2A and FIG. 2B. The health monitoring device is applied to an earphone. In this embodiment, the optical monitoring module 2 is disposed inside the main body 11 of the wearable component 1. The driving controller 21 includes a driving circuit board 211 and a microprocessor 212. The driving circuit board 211 is configured and positioned inside the main body 11 of the wearable component 1, and the driving circuit board 211 is under the earphone soundbox 12. The optical sensor 22, each light-emitting element 23 and the microprocessor 212 are packaged and positioned on the driving circuit board 211. The optical sensor 22, each light-emitting element 23 and the microprocessor 212 are connected to the driving circuit board 211 for receiving the required electrical connection and driving control signal. Moreover, the optical sensor 22 and the light-emitting element 23 are corresponding in position to the monitoring opening 111 of the main body 11. Therefore, the light source emitted by the light-emitting element 23 irradiates on the skin tissue 4 of the subject through the monitoring opening 111, and the optical sensor 22 receives the reflection light and generates a sensing signal accordingly for monitoring. The microprocessor 212 of the driving controller 21 converts the sensing signal into health data information and outputs the health data information. In addition, the gas-collecting actuator 31 is disposed on and connected to the driving circuit board 211 for receiving the required electrical connection and driving control signal. The elastic air bag 32 is disposed outside the main body 11 of the wearable component 1 and surrounds the exterior of the earphone soundbox 12. The elastic air bag 32 is in fluid communication with the gas-collecting actuator 31 through a gas connection channel 33. Accordingly, the gas-collecting actuator 31 transports the gas to the interior of the elastic air bag 32, and the elastic air bag 32 is inflated and elastically protrudes out of the wearable component 1 (as shown in FIG. 2B). Therefore, the wearable component 1 can be securely worn on the body part of the subject, and the optical sensor 22 is allowed to be attached on the skin tissue 4 of the subject (as shown in FIG. 11) for accurately monitoring the health data information.

Please refer to FIGS. 3 to 6. The health monitoring device is applied to the wearable ring. In this embodiment, the wearable component 1 includes a ring structure 13 connected to the exterior of the main body 11. The ring structure 13 is for example but not limited to be composed of a soft or rigid material, such as a silicone material, a plastic material, a metal material or other related materials. The wearable component 1 is mainly used to wrap around a specific part (e.g., wrist or an ankle) of the subject, but not limited thereto. As to the connection manner of the two ends of the ring structure 13, the attaching means of Velcro may be applied. In an embodiment, the fastening means of the convex-and-concave butt joints, or the buckle ring commonly used for the general wearable component may be applied. In other embodiment, the ring structure 13 may be integrally formed from one piece. The connection manner is adjustable according to the practical requirements. The present disclosure is not limited thereto.

The elastic air bag 32 is disposed on the inner surface of the ring structure 13. The optical monitoring module 2 is disposed inside the main body 11 of the wearable component 1. The driving controller 21 includes a driving circuit board 211 and a microprocessor 212. The driving circuit board 211 is configured and positioned inside the main body of the wearable component 1. The optical sensor 22, each light-emitting element 23 and the microprocessor 212 are packaged and are positioned on the driving circuit board 211. The optical sensor 22, each light-emitting element 23 and the microprocessor 212 are connected to the driving circuit board 211 for receiving the required electrical connection and driving control signal. Moreover, the optical sensor 22 and the light-emitting element 23 are corresponding in position to the monitoring opening 111 of the main body 11. Therefore, the light source emitted by the light-emitting element 23 irradiates on the skin tissue 4 of the subject through the monitoring opening 111, and the optical sensor 22 receives the reflection light and generates a sensing signal accordingly for monitoring. The microprocessor 212 of the driving controller 21 converts the sensing signal into health data information and outputs the health data information.

In addition, an embedding seat 112 is disposed inside the main body 11. A gas-collecting slot opening 113 and a communication channel 114 are disposed on the bottom of the embedding seat 112. The gas-collecting slot opening 113 and the communication channel 114 are in fluid communication with each other, and the communication channel 114 is in fluid communication with the elastic air bag 32. A cover plate 115 is disposed on the bottom of the main body 11 for covering and sealing the gas-collecting slot opening 113 and the communication channel 114. The gas-collecting actuator 31 is positioned in the embedding seat 112, and the gas-collecting actuator 31 is connected to the driving circuit board 211 for receiving the required electrical connection and driving control signal. The position of gas collecting of the gas-collecting actuator 31 is in fluid communication with the gas-collecting slot opening 113 and is sealed. Accordingly, the gas-collecting actuator 31 transports the gas to the gas-collecting slot opening 113 and the communication channel 114, and the gas flows into the interior of the elastic air bag 32. The elastic air bag 32 is inflated and elastically protrudes out of the wearable component 1 (as shown in FIG. 6). Therefore, the wearable component 1 can be securely worn on the body part of the subject, and the optical sensor 22 is allowed to be attached on the skin tissue 4 of the subject (as shown in FIG. 11) for accurately monitoring the health data information.

For the health monitoring device of the present disclosure, the wearable component 1 can be securely worn on the body part of the subject by the gas-collecting actuator 31 transporting the gas to the interior of the elastic air bag 32. Further, the optical sensor 22 is allowed to be attached on the skin tissue 4 of the subject for accurately monitoring the health data information. The structure and actions for supplying of the gas-collecting actuator 31 are described as follows.

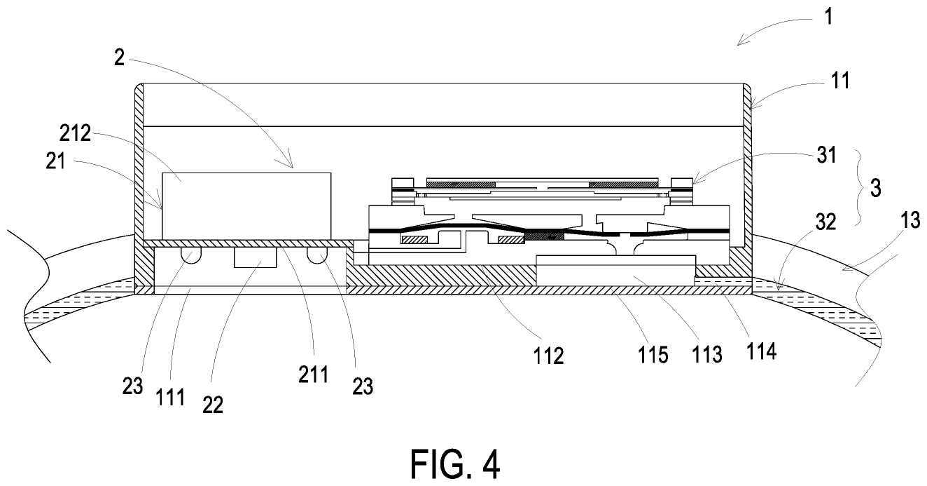

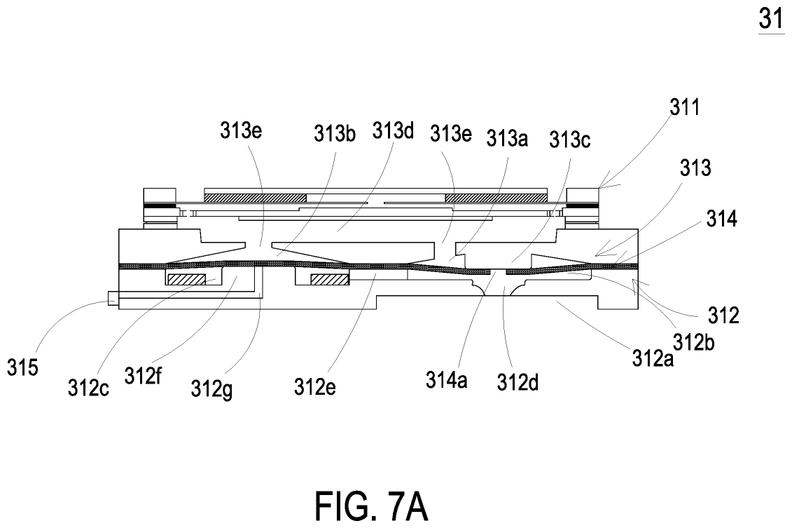

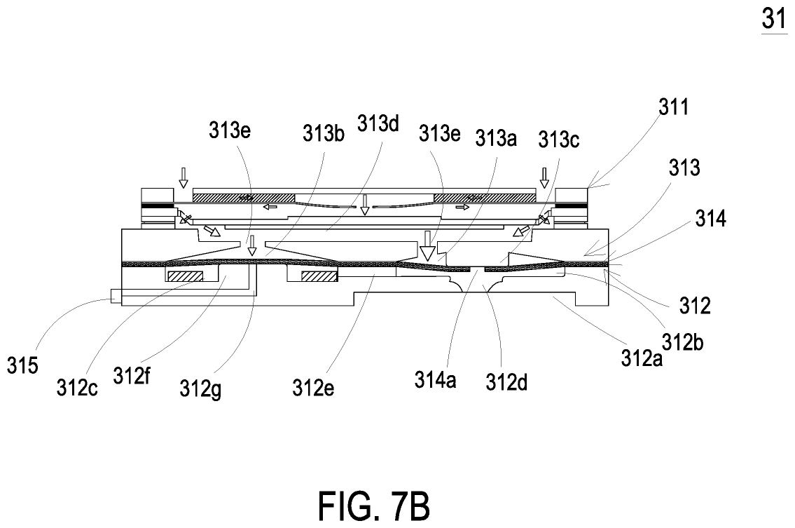

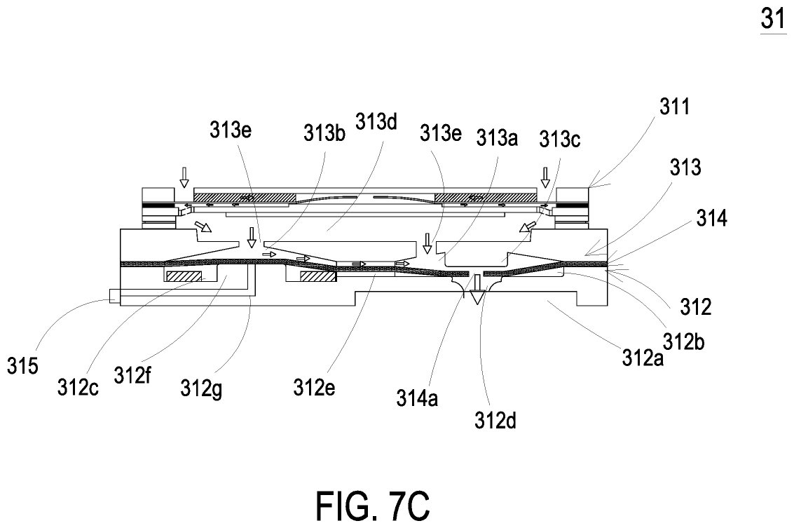

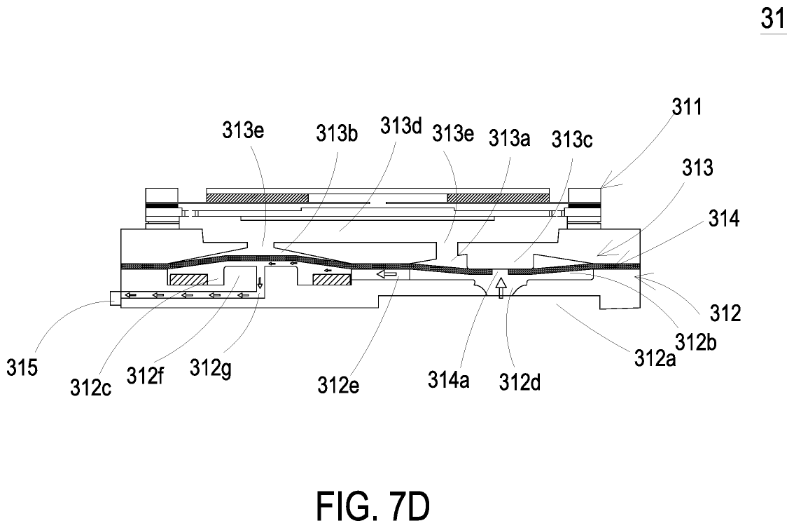

Please refer to FIG. 2A, FIG. 4 and FIGS. 7A to 7D. The gas-collecting actuator 31 includes a micro pump 311, a gas-collector seat 312, a chamber plate 313, a valve membrane 314 and a valve switch 315. The gas-collector seat 312 is disposed on the driving circuit board 211 (as shown in FIG. 2A) or is positioned in the embedding seat 112 (as shown in FIG. 4). The gas-collector seat 312 includes a gas-collecting slot 312a concavely formed on a bottom surface of the gas-collector seat 312. The gas-collector seat 312 further includes a lower gas-collecting chamber 312b and a lower pressure-releasing chamber 312c formed on a top surface of the gas-collector seat 312. In the embodiment, a gas-collecting perforation 312d is formed between the gas-collecting slot 312a and the lower gas-collecting chamber 312b for allowing the gas-collecting slot 312a and the lower gas-collecting chamber 312b to communicate with each other. The lower gas-collecting chamber 312b and the lower pressure-releasing chamber 312c are separated apart on the top surface of the gas-collector seat 312. A communication channel 312e is disposed between the lower gas-collecting chamber 312b and the lower pressure-releasing chamber 312c for allowing the lower gas-collecting chamber 312b and the lower pressure-releasing chamber 312c to communicate with each other. In the embodiment, a first protrusion 312f is formed in the lower pressure-releasing chamber 312b, and a pressure-releasing perforation 312g is disposed at a center of the first protrusion 312E The pressure-releasing perforation 312g is in fluid communication with the lower pressure-releasing chamber 312c. Moreover, the pressure-releasing perforation 312g is in fluid communication with the valve switch 315, and the valve switch 315 is configured for controlling the exhausting of the pressure-releasing perforation 312g. As shown in FIG. 2A, the gas-collecting slot 312a is in fluid communication with the gas connection channel 33 and is sealed. Therefore, the elastic air bag 32 is in fluid communication with the gas-collecting slot 312a and the gas-collecting perforation 312d, so that the elastic air bag 32 is capable of being inflated and elastically protruding out of the main body 11. Alternatively, as shown in FIG. 4, the gas-collecting slot 312a is in fluid communication with the gas-collecting slot opening 113 and is sealed. Therefore, the communication channel 114, the elastic air bag 32 and the gas-collecting slot 312a are in fluid communication with each other, so that the elastic air-bag 32 is capable of being inflated and elastically protruding out of the main body 11. In the embodiment, the chamber plate 313 is carried and disposed on the gas-collector seat 312. The chamber plate 313 includes an upper gas-collecting chamber 313a and an upper pressure-releasing chamber 313b formed on a top surface spatially corresponding to the gas-collector seat 312. The upper gas-collecting chamber 313a and the lower gas-collecting chamber 312b are matched and sealed with each other. The upper pressure-releasing chamber 313b and the lower pressure-releasing chamber 312c are matched and sealed with each other. A second protrusion 313c is formed in the upper gas-collecting chamber 313a. A communication chamber 313d is concavely formed on a bottom surface of the chamber plate 313 opposite to the upper gas-collecting chamber 313a and the upper pressure-releasing chamber 313b. The micro pump 311 is carried and disposed on the chamber plate 313 to seal and cover the communication chamber 313d. The communication chamber 313d is in fluid communication with the upper gas-collecting chamber 313a and the upper pressure-releasing chamber 313b respectively via at least one communication aperture 313e. Moreover, the valve membrane 314 is disposed between the gas-collector seat 312 and the chamber plate 313, and the valve membrane 314 is abutted against the first protrusion 312f to seal the pressure-releasing perforation 312g. The valve membrane 314 has a valve aperture 314a disposed at a position abutted against the second protrusion 313c, and the valve aperture 314a is sealed when the valve membrane 314 abuts against the second protrusion 313c. That is, the valve aperture 314a is located at the position where the valve membrane 314 abuts against the second protrusion 313c.

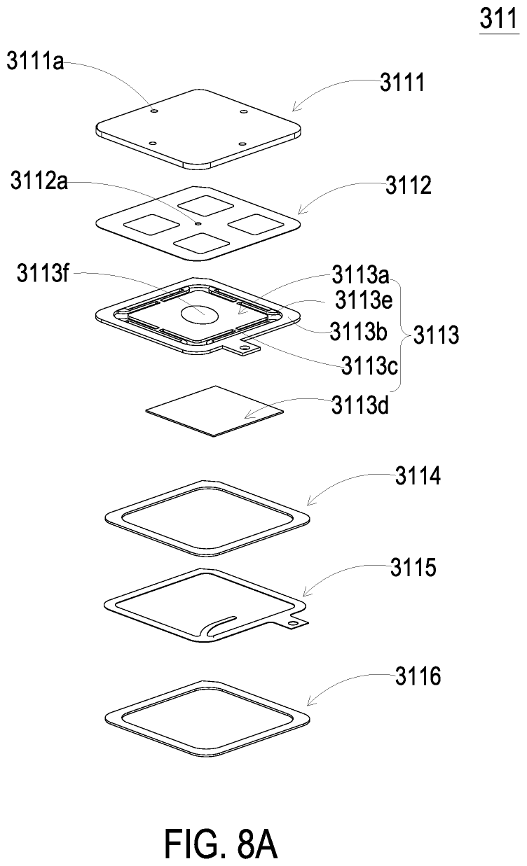

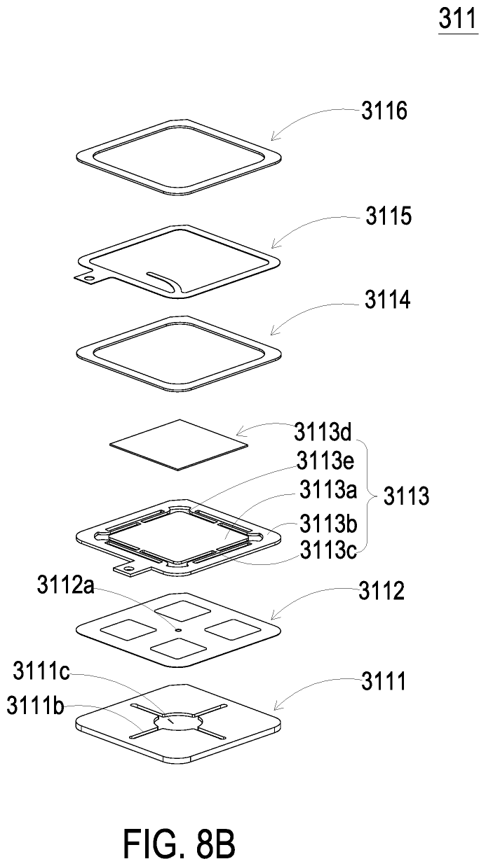

Please refer to FIGS. 8A and 8B. The micro pump 311 includes a gas inlet plate 3111, a resonance plate 3112, a piezoelectric actuator 3113, a first insulation plate 3114, a conducting plate 3115 and a second insulation plate 3116 stacked sequentially. The gas inlet plate 3111 has at least one inlet aperture 3111a, at least one convergence channel 3111b and a convergence chamber 3111c. The inlet aperture 3111a allows a gas to flow therein. The convergence channel 3111b is disposed correspondingly to the inlet aperture 3111a and guides the gas from the inlet aperture 3111a toward the convergence chamber 3111c. In the embodiment, the number of the inlet apertures 3111a and the number of the convergence channels 3111b are the same. Preferably but not exclusively, there are four inlet apertures 3111a and four convergence channels 3111b. The four inlet apertures 3111a are in fluid communication with the four convergence channels 3111b, respectively, and the four convergence channels 3111b guide the gas to the convergence chamber 3111c.

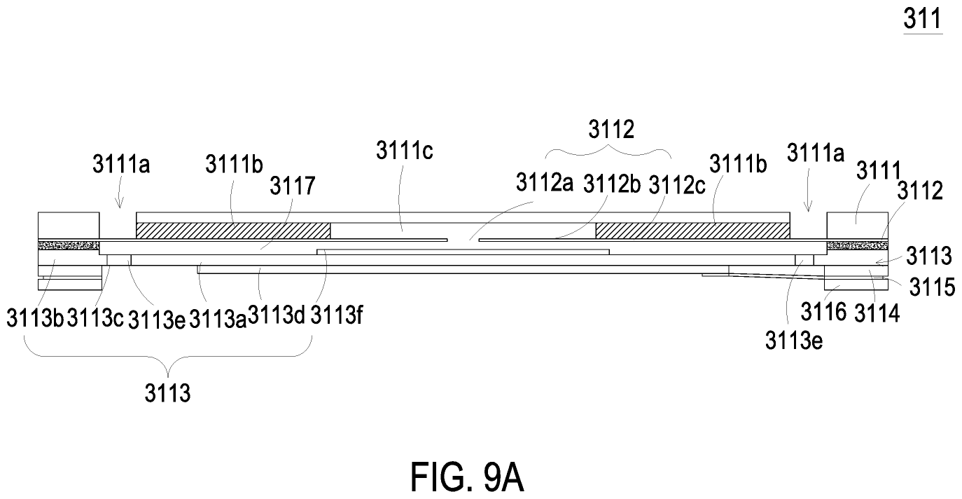

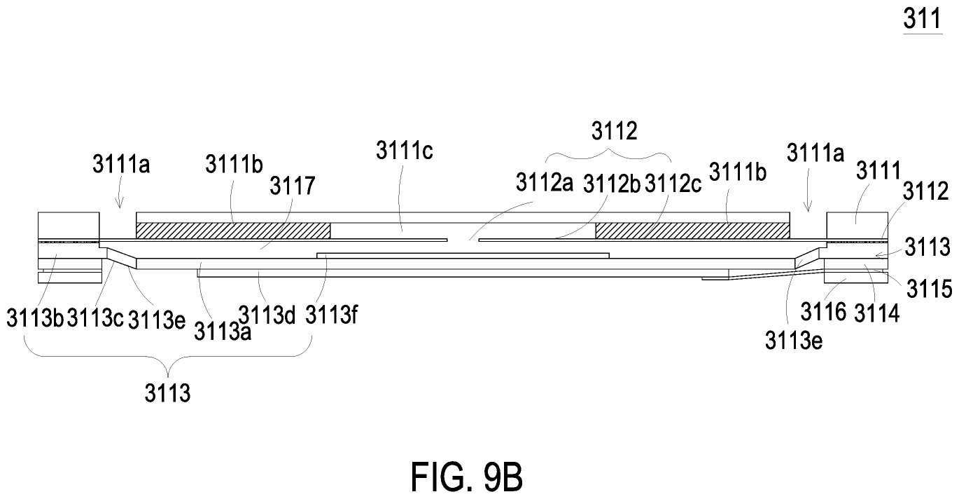

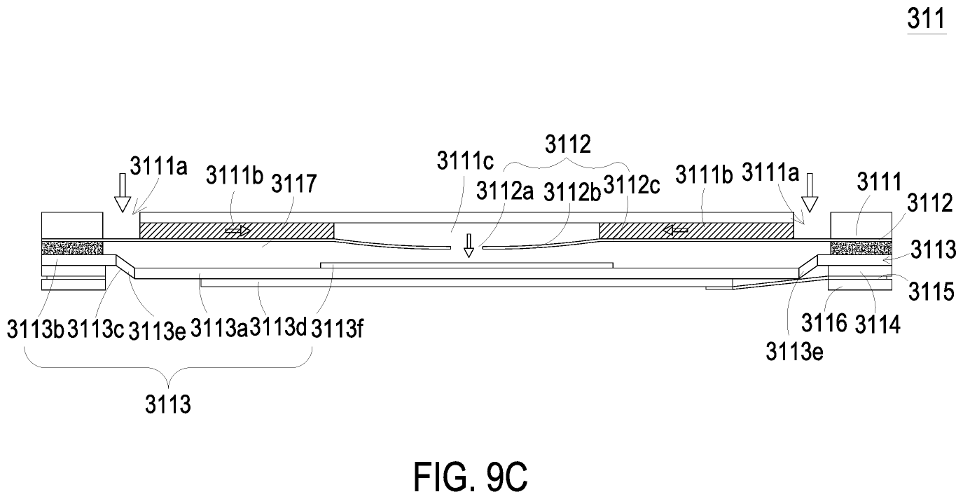

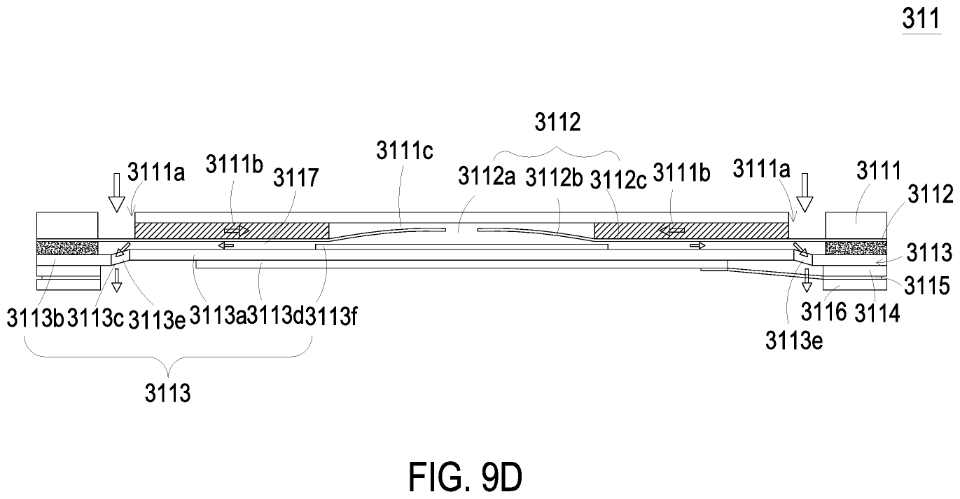

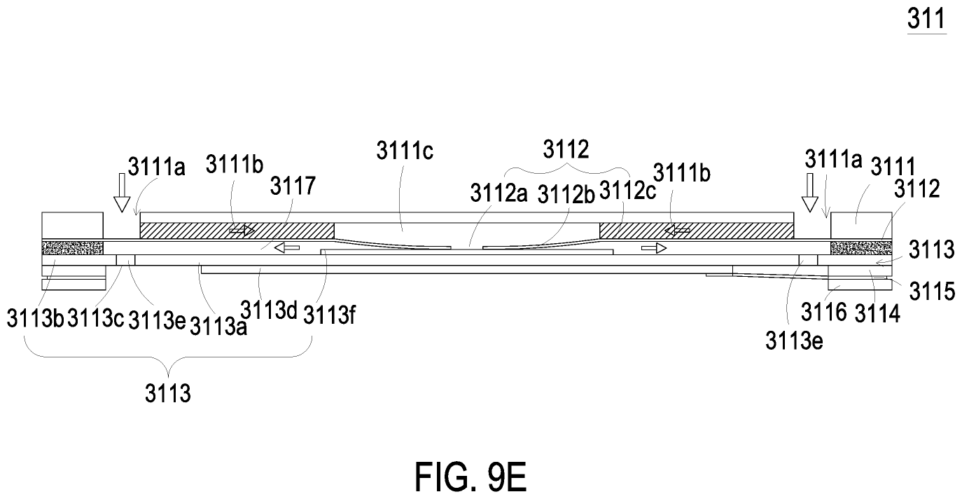

Please refer to FIGS. 8A, 8B and 9A. In the embodiment, the resonance plate 3112 is assembled with the gas inlet plate 3111 by means of adhesion. The resonance plate 3112 has a central aperture 3112a, a movable part 3112b and a fixing part 3112c. The central aperture 3112a is disposed at a center of the resonance plate 3112 and is aligned with the convergence chamber 3111c of the gas inlet plate 3111. The movable part 3112b surrounds the central aperture 3112a and spatially corresponds to the convergence chamber 3111c. The fixing part 3112c is located at a peripheral portion of the resonance plate 3112 and is attached on the gas inlet plate 3111.

Please continue referring to FIGS. 8A, 8B and 9A. In the embodiment, the piezoelectric actuator 3113 includes a suspension plate 3113a, an outer frame 3113b, at least one bracket 3113c, a piezoelectric element 3113d, at least one vacant space 3113e and a bulge 3113E Preferably but not exclusively, the suspension plate 3113a is a square suspension plate. Compared with the design of the circular suspension plate, the square structure of the suspension plate 3113a obviously has the advantage of power saving. The power consumption of the capacitive load operating at the resonant frequency is increased as the frequency is increased, and the resonance frequency of the suspension plate 3113a in side-long square type is obviously lower than that of the circular suspension plate. Accordingly, the relative power consumption of the square suspension plate is obviously lower than that of circular suspension plate. Therefore, the suspension plate 3113a of the present disclosure, which is designed in a square type, has advantage of power saving.

In the embodiment, the outer frame 3113b is arranged around the suspension plate 3113a. The at least one bracket 3113c is connected between the suspension plate 3113a and the outer frame 3113b for elastically supporting the suspension plate 3113a. In the embodiment, a length of a side of the piezoelectric element 3113d is smaller than or equal to a length of a side of the suspension plate 3113a. The piezoelectric element 3113d is attached on a surface of the suspension plate 3113a for driving the suspension plate 3113a to undergo the bending vibration in response to an applied voltage. The at least one vacant space 3113e is formed among the suspension plate 3113a, the outer frame 3113b and the bracket 3113c for allowing the gas to flow therethrough. In the embodiment, the suspension plate 3113a has a first surface attached on the piezoelectric element 3113d and a second surface opposite to the first surface, and the bulge 3113f is disposed on the second surface. In the embodiment, the bulge 3113f and the suspension plate 3113a are integrally formed from one piece, that is, the bulge 3113f is formed by an etching process, and a convex structure is integrally formed on the second surface.

Please continue referring to FIGS. 8A, 8B and 9A. The gas inlet plate 3111, the resonance plate 3112, the piezoelectric actuator 3113, the first insulation plate 3114, the conducting plate 3115 and the second insulation plate 3116 are stacked sequentially. A chamber space 3117 is formed between suspension plate 3113a and the resonance plate 3112. Preferably but not exclusively, a filler (e.g., a conductive adhesive) is filled in a gap generated between the resonance plate 3112 and the outer frame 3113b of the piezoelectric actuator 3113, so that a specific depth between the resonance plate 3112 and the suspension plate 3113a can be maintained to form the chamber space 3117. Thus, the gas can be introduced to flow more rapidly. Moreover, since the proper distance between the suspension plate 3113a and the resonance plate 3112 is maintained, the contact interference and the generated noise are largely reduced. In some embodiments, alternatively, the height of the outer frame 3113b of the piezoelectric actuator 3113 is increased. Accordingly, the thickness of the conductive adhesive filled within the gap between the resonance plate 3112 and the outer frame 3113b of the piezoelectric actuator 3113 is reduced. Therefore, in the case where the suspension plate 3113a and the resonance plate 3112 are maintained at a proper distance, the thickness of the conductive adhesive filled within the overall assembly of the micro pump 311 won't be affected by a hot pressing temperature and a cooling temperature. It benefits to avoid that the conductive adhesive affects the actual size of the chamber space 3117 due to the factors of thermal expansion and contraction after the assembly is completed. The present disclosure is not limited thereto. In addition, since the transportation efficiency of the micro pump 311 is affected by the chamber space 3117, it is important to maintain a fixed-size chamber space 3117 for providing stable transportation efficiency of the micro pump 311.

As shown in FIG. 9B, in another exemplary structure of the piezoelectric actuator 3113, the suspension plate 3113a can be formed by a stamping method. The stamping method makes the suspension plate 3113a extended outwardly at a distance. The distance extended outwardly may be adjusted by the bracket 3113c formed between the suspension plate 3113a and the outer frame 3113b, so that a surface of the bulge 3113f on the suspension plate 3113a is not coplanar with a surface of the outer frame 3113b. A small amount of filling material (e.g., conductive adhesive) is applied to the assembly surface of the outer frame 3113b for attaching the piezoelectric actuator 3113 on the fixing part 3112c of the resonance plate 3112 by means of hot pressing. Further, the piezoelectric actuator 3113 is assembled with the resonance plate 3112. In this way, the entire structure may be improved by adopting the stamping method to form the suspension plate 3113a of the piezoelectric actuator 3113, thereby modifying the chamber space 3117. A desired size of the chamber space 3117 may be satisfied by simply adjusting the distance from resonance plate 3112 to the suspension plate 3113a of the piezoelectric actuator 3113 through the stamping method. It simplifies the structural design for adjusting the chamber space 3117. At the same time, it achieves the advantages of simplifying the process and saving the process time. In the embodiment, the first insulation plate 3114, the conducting plate 3115 and the second insulation plate 3116 are all frame-shaped thin sheet and are stacked sequentially on the piezoelectric actuator 3113 to obtain the entire structure of the micro pump 311.

For describing the actions of the micro pump 311, please refer to FIGS. 9C to 9E. Firstly, as shown in FIG. 9C, when the piezoelectric element 3113d of the piezoelectric actuator 3113 is deformed in response to an applied voltage, the suspension plate 3113a is displaced in a direction away from the gas inlet plate 3111. In that, the volume of the chamber space 3117 is increased, a negative pressure is formed in the chamber space 3117, and the gas in the convergence chamber 3111c is inhaled into the chamber space 3117. At the same time, the resonance plate 3112 is in resonance and thus displaced synchronously in the direction away from the gas inlet plate 3111. Thereby, the volume of the convergence chamber 3111c is increased. Since the gas in the convergence chamber 3111c flows into the chamber space 3117, the convergence chamber 3111c is also in a negative pressure state, and the gas is sucked into the convergence chamber 3111c by flowing through the inlet aperture 3111a and the convergence channel 3111b. Then, as shown in FIG. 9D, the piezoelectric element 3113d drives the suspension plate 3113a to be displaced toward the gas inlet plate 3111 to compress the chamber space 3117. Similarly, the resonance plate 3112 is actuated by the suspension plate 3113a (i.e., in resonance with the suspension plate 3113a) and is displaced toward the gas inlet plate 3111. Thus, the gas in the chamber space 3117 is compressed synchronously and forced to be further transported through the vacant space 3113e to achieve the effect of gas transportation. Finally, as shown in FIG. 9E, when the suspension plate 3113a is vibrated back to the initial state, which is not driven by the piezoelectric element 3113d, the resonance plate 3112 is also driven to displace in the direction away from the gas inlet plate 3111 at the same time. In that, the resonance plate 3112 pushes the gas in the chamber space 3117 toward the vacant space 3113e, and the volume of the convergence chamber 3111c is increased. Thus, the gas can continuously flow through the inlet aperture 3111a and the convergence channel 3111b and be converged in the confluence chamber 3111c. By repeating the actions of the micro pump 311 shown in the above-mentioned FIGS. 9C to 9E continuously, the micro pump 311 can continuously transport the gas at a high speed to accomplish the gas transportation and output operations of the micro pump 311.

Please refer to FIG. 9A. In the embodiment, the gas inlet plate 3111, the resonance plate 3112, the piezoelectric actuator 3113, the first insulation plate 3114, the conducting plate 3115 and the second insulation plate 3116 of the micro pump 311 are all produced by a micro-electromechanical surface micromachining technology. Thereby, the volume of the micro pump 311 is reduced, and a micro-electromechanical system (MEMS) micro pump 311 is constructed.

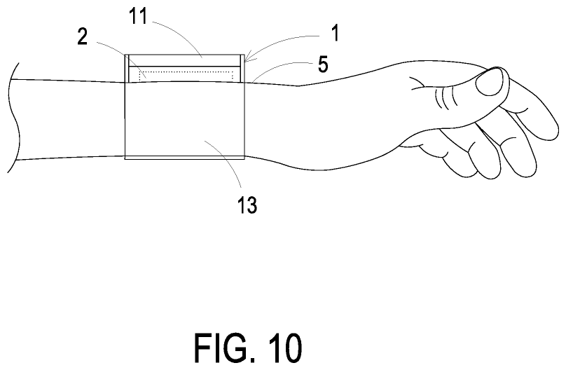

According to the above descriptions, as shown in FIG. 10, the health monitoring device implemented as being worn on the wrist. Under this circumstance, the gas-collecting actuator 31 is implemented as shown in FIGS. 7B and 7C. When the gas-collecting actuator 311 is controlled and driven to transport a gas, the gas is inhaled from outside of the gas-collecting actuator 31 and transported to the communication chamber 313d by the micro pump 311. Then, the gas is transported from the communication chamber 313d to the upper gas-collecting chamber 313a and the upper gas-releasing chamber 313b through the communication aperture 313e. Consequently, the valve membrane 314 is pushed to move apart from the second protrusion 313c. The valve membrane 314 is pushed to abut against the first protrusion 312f and to seal the pressure-releasing perforation 312g. Meanwhile, the gas in the upper pressure-releasing chamber 313b is transported into the upper gas-collecting chamber 313a through the communication channel 312e and further transported into the lower gas-collecting chamber 312b of the gas-collector seat 312 through the valve aperture 314a of the valve membrane 314. Afterwards, the gas is converged to the elastic air bag 32 (as shown in FIG. 4) in fluid communication with the gas-collecting perforation 312d, and the elastic air bag 32 is inflated and elastically protrudes out of the main body 11 of the wearable component 1. Accordingly, the wearable component 1 is securely worn on the body part of the subject, and the optical sensor 22 is allowed to be attached on the skin tissue 4 of the subject (as shown in FIG. 11). In this way, the elastic air bag 32 is inflated and is abutted against the skin tissue 4 of the subject, and the blood vessel 5 between the skin tissue 4 and the bone 6 of the subject is pressed to stop blood flow. Consequently, the optical sensor 22 is able to accurately monitor the health data information.

Certainly, if the health monitoring device of the present disclosure is not worn on the user, the inflating operation is stopped. As shown in FIG. 7D, the gas-collecting actuator 31 stops transporting gas. Under this circumstance, the gas pressure inside the elastic air bag 32 is greater than that of the communication chamber 313d. The gas converged in the elastic air bag 32 pushes the valve membrane 314 to move and abut against the second protrusion 313c, and the valve aperture 314a is sealed. Meanwhile, the gas pushes the valve membrane 314 to move apart from the first protrusion 312f for opening the pressure-releasing perforation 312g. The gas converged in the elastic air bag 32 is transported to the pressure-releasing perforation 312g through the communication channel 312e. Further, the valve switch 315 is controlled to open for controlling the exhausting of the pressure-releasing perforation 312g, thus the gas in the elastic air bag 32 is discharged out of the gas-collecting actuator 31. Consequently, the pressure-releasing operation of the elastic air bag 32 is completed.

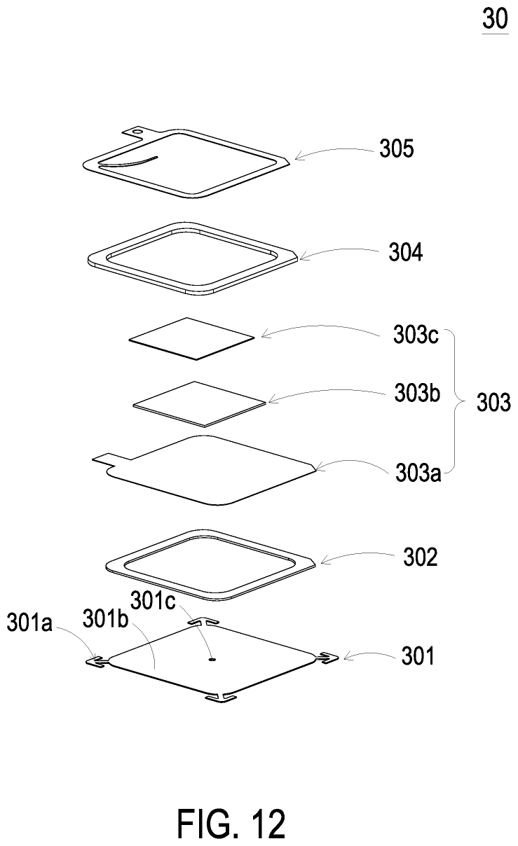

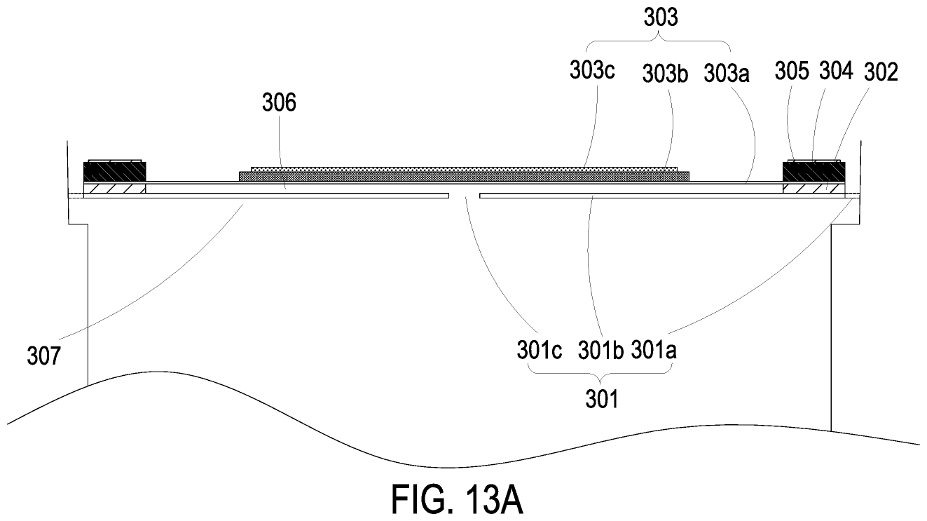

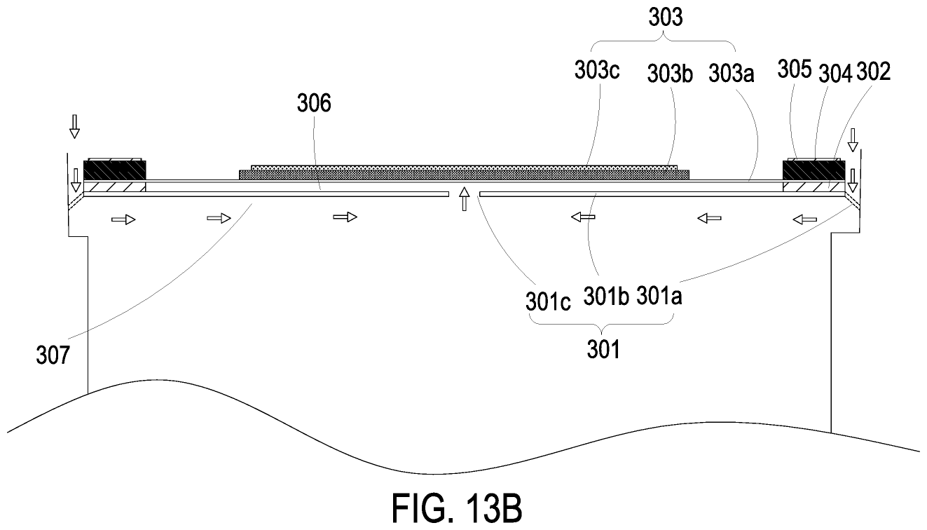

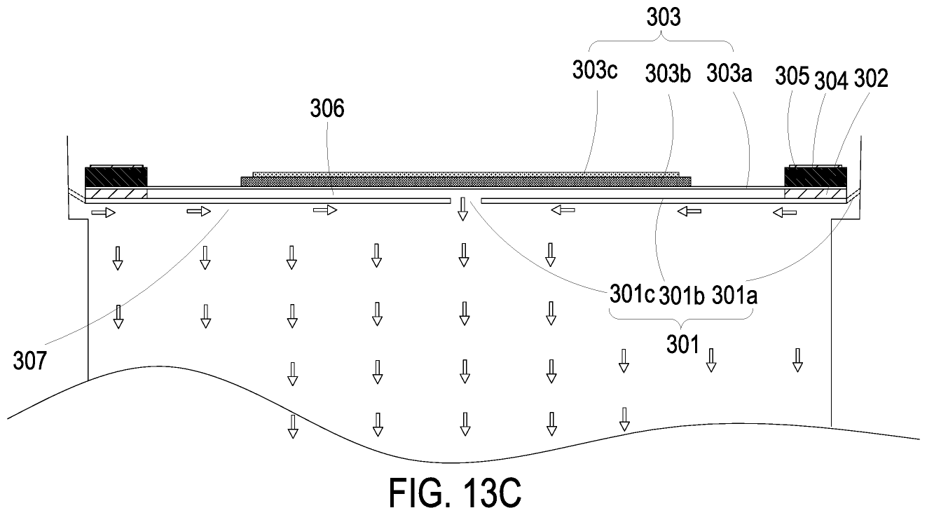

In addition to the micro pump 311 described above, the gas-collecting actuator 31 may be operated with a micro box pump 30 to implement gas transportation. Please refer to FIG. 12 and FIGS. 13A to 13C. The micro box pump 30 includes a nozzle plate 301, a chamber frame 302, an actuating element 303, an insulation frame 304 and a conducting frame 305, which are stacked on each other sequentially. The nozzle plate 301 includes a plurality of connecting element 301a, a suspension board 301b and a central aperture 301c. The suspension board 301b is permitted to bend and vibrate. The plurality of connecting elements 301a is connected to the edge of the suspension board 301b. In this embodiment, there are four connecting elements 301a, which are connected to four corners of the suspension board 301b respectively, but not limited thereto. The central aperture 301c is formed in the center of the suspension board 301b. The chamber frame 302 is carried and stacked on the suspension board 301b. The actuating element 303 is carried and stacked on the chamber frame 302, and includes a piezoelectric carrying plate 303a, an adjusting resonance plate 303b and a piezoelectric plate 303c. The piezoelectric carrying plate 303a is carried and stacked on the chamber frame 302. The adjusting resonance plate 303b is carried and stacked on the piezoelectric carrying plate 303a. The piezoelectric plate 303c is carried and stacked on the adjusting resonance plate 303b. As the piezoelectric plate 303c is actuated by an applied voltage, the piezoelectric plate 303c deforms to drive the piezoelectric carrying plate 303a and the adjusting resonance plate 303b to bend and vibrate in a reciprocating manner. The insulation frame 304 is carried and stacked on the piezoelectric carrying plate 303a of the actuating element 303. The conducting frame 305 is carried and the stacked on the insulation frame 304. A resonance chamber 306 is formed among the actuating element 303, the chamber frame 302 and the suspension board 301b.

Please refer to FIG. 12 and FIGS. 13A to 13C again. FIGS. 13A to 13C schematically illustrate the actions of the micro box pump 30 of present disclosure. First, please refer to FIG. 12 and FIG. 13A. The micro box pump 30 is securely disposed via the plurality of connecting elements 301a. An airflow chamber 307 is formed under the bottom of the nozzle plate 301. Then, please refer to FIG. 13B. When the piezoelectric plate 303c of the actuating element 303 is actuated by an applied voltage, the piezoelectric plate 303c is subjected to a deformation owing to the piezoelectric elect, and the adjusting resonance plate 303b and the piezoelectric carrying plate 303a are driven to vibrate synchronously. Meanwhile, the nozzle plate 301 is driven to move owing to the Helmholtz resonance effect, and the actuating element 303 moves in a direction away from the nozzle plate 301. Since the actuating element 303 moves in a direction away from the nozzle plate 301, the volume of the airflow chamber 307 at the bottom of the nozzle plate 301 is increased, and a negative pressure is formed in the airflow chamber 307. The air outside the micro box pump 30 is inhaled into the airflow chamber 307 through the vacant spaces among the plurality of connecting elements 301a of the nozzle plate 301 due to the pressure gradient, and is further compressed. Finally, please refer to FIG. 13C. The gas flows into the airflow chamber 307 continuously, and a positive pressure is formed in the airflow chamber 307. Meanwhile, the actuating element 303 is driven to vibrate in a direction toward the nozzle plate 301 in response to the voltage, and the volume of the airflow chamber 307 is compressed. The gas in the airflow chamber 307 is pushed and is discharged from the micro box pump 30. Consequently, the gas transportation is implemented.

In an embodiment, the micro box pump 30 is a micro-electromechanical system gas pump produced by micro-electromechanical manufacturing process. The nozzle plate 301, the chamber frame 302, the actuating element 303, the insulation frame 304 and the conducting frame 305 are all produced by a micro-electromechanical surface micromachining technology. Thereby, the volume of the micro box pump 30 is reduced.

From the above descriptions, the present disclosure provides a health monitoring device. An air-bag positioning assembly, which includes a gas-collecting actuator and an elastic air bag, is configured for securely disposing and positioning. The gas-collecting actuator transports the gas to the interior of the air bag. Therefore, the wearable component is securely disposed on the body part of the subject, and the optical sensor is attached on skin tissue of the subject for accurately monitoring the health information. The present invention is industrially valuable.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A health monitoring device, comprising:
a wearable component worn on a body part of a subject and contacted with a skin tissue thereof, wherein the wearable component has a main body, and the main body has a monitoring opening;
an optical monitoring module disposed inside the main body of the wearable component and comprising a driving controller, an optical sensor and at least one light-emitting element, wherein the optical sensor and the light-emitting element are disposed corresponding in position to the monitoring opening, a light source emitted by the light-emitting element irradiates on the skin tissue, and the optical sensor receives a reflection light and generates a sensing signal accordingly, whereby the driving controller converts the sensing signal into health data information and outputs the health data information; and
an air-bag positioning assembly comprising a gas-collecting actuator and an elastic air bag, wherein the gas-collecting actuator is disposed inside the main body of the wearable component, the elastic air bag is disposed on the wearable component, the gas-collecting actuator transports the gas to the interior of the elastic air bag, and the elastic air bag is inflated and elastically protrudes out of the wearable component, whereby the wearable component is securely worn on the body part of the subject, and allows the optical sensor to be attached on the skin tissue of the subject for accurately monitoring the health data information.

2. The health monitoring device according to claim 1, wherein the driving controller comprises a driving circuit board and a microprocessor, wherein the driving circuit board is configured and positioned inside the main body of the wearable component, and the optical sensor, the light-emitting element and the microprocessor are packaged and positioned on the driving circuit board and are connected to the driving circuit board for receiving a required electrical connection and driving control signal, and wherein the gas-collecting actuator is connected to the driving circuit board for receiving a required electrical connection and driving control signal, and the microprocessor converts the sensing signal generated by the optical sensor into the health data information and outputs the health data information.

3. The health monitoring device according to claim 1, wherein the health data information comprises a heart rate data, an electrocardiogram data and a blood pressure data.

4. The health monitoring device according to claim 1, wherein the gas-collecting actuator comprises a micro pump, a gas-collector seat, a chamber plate, a valve membrane and a valve switch, wherein the gas-collector seat is carried and disposed in the main body, the gas-collector seat comprises a gas-collecting slot concavely formed on a bottom surface of the gas-collector seat, the gas-collector seat further comprises a lower gas-collecting chamber and a lower pressure-releasing chamber formed on a top surface of the gas-collector seat, a gas-collecting perforation is formed between the gas-collecting slot and the lower gas-collecting chamber for allowing the gas-collecting slot and the lower gas-collecting chamber to communicate with each other, the lower gas-collecting chamber and the lower pressure-releasing chamber are separated apart on the top surface of the gas-collector seat, a communication channel is disposed between the lower gas-collecting chamber and the lower pressure-releasing chamber for allowing the lower gas-collecting chamber and the lower pressure-releasing chamber to communicate with each other, a first protrusion is formed in the lower pressure-releasing chamber, a pressure-releasing perforation is disposed at a center of the first protrusion, the pressure-releasing perforation is in fluid communication with the lower pressure-releasing chamber, the pressure-releasing perforation is in fluid communication with the valve switch, the valve switch is configured for controlling the exhausting of the pressure-releasing perforation, the elastic air bag is in fluid communication with the gas-collecting slot and the gas-collecting perforation, the chamber plate is carried and disposed on the gas-collector seat, the chamber plate comprises an upper gas-collecting chamber and an upper pressure-releasing chamber formed on a top surface of the chamber plate spatially corresponding to the gas-collector seat, the upper gas-collecting chamber and the lower gas-collecting chamber are matched and sealed with each other, the upper pressure-releasing chamber and the lower pressure-releasing chamber are matched and sealed with each other, a second protrusion is formed in the upper gas-collecting chamber, a communication chamber is concavely formed on a bottom surface of the chamber plate opposite to the upper gas-collecting chamber and the upper pressure-releasing chamber, the micro pump is carried and disposed on the chamber plate to seal and cover the communication chamber, the communication chamber is in fluid communication with the upper gas-collecting chamber and the upper pressure-releasing chamber respectively via at least one communication aperture, the valve membrane is disposed between the gas-collector seat and the chamber plate, the valve membrane is abutted against the first protrusion to seal the pressure-releasing perforation, the valve membrane has a valve aperture disposed at a position where the valve membrane abuts against the second protrusion, and the valve aperture is sealed by the second protrusion.

5. The health monitoring device according to claim 4, wherein the micro pump is controlled to transport a gas to the communication chamber, then the gas is transported from the communication chamber to the upper gas-collecting chamber and the upper gas-releasing chamber through the communication aperture, the valve membrane is pushed to move apart from the second protrusion, the valve membrane is pushed to abut against the first protrusion and to seal the pressure-releasing perforation, the gas in the upper pressure-releasing chamber is transported into the upper gas-collecting chamber through the communication channel and further transported into the lower gas-collecting chamber through the valve aperture of the valve membrane, the gas is converged to the gas-collecting slot and the elastic air bag through the gas-collecting perforation, the elastic air bag is inflated and elastically protrudes out of the main body of the wearable component, and the wearable component is securely worn on the body part of the subject.

6. The health monitoring device according to claim 4, wherein when the micro pump stops transporting the gas, the gas pressure inside the elastic air bag is greater than that of the communication chamber, the gas converged in the elastic air bag pushes the valve membrane to move and abut against the second protrusion, the valve aperture is sealed, the gas pushes the valve membrane to move apart from the first protrusion for opening the pressure-releasing perforation, the valve switch is controlled to open for controlling the exhausting of the pressure-releasing perforation, the gas in the elastic air bag is discharged out of the gas-collecting actuator, through the communication channel and the pressure-releasing perforation, and the pressure-releasing operation of the elastic air bag is completed.

7. The health monitoring device according to claim 4, wherein the micro pump comprises:
a gas inlet plate having at least one inlet aperture, at least one convergence channel and a convergence chamber, wherein the inlet aperture allows a gas to flow in, the convergence channel is disposed correspondingly to the inlet aperture and guides the gas from the inlet aperture toward the convergence chamber;
a resonance plate assembled with the gas inlet plate and having a central aperture, a movable part and a fixing part, wherein the central aperture is disposed at a center of the resonance plate and is aligned with the convergence chamber of the gas inlet plate, the movable part surrounds the central aperture and spatially corresponds to the convergence chamber, and the fixing part is located at a peripheral portion of the resonance plate and is attached on the gas inlet plate; and
a piezoelectric actuator assembled with and disposed corresponding to the resonance plate,
wherein a chamber space is formed between the resonance plate and the piezoelectric actuator, when the piezoelectric actuator is driven, the gas is introduced into the at least one inlet aperture of the gas inlet plate, converged to the convergence chamber along the at least one convergence channel, and flows into the central aperture of the resonance plate, whereby the gas is further transported through a resonance between the piezoelectric actuator and the movable part of the resonance plate.

8. The health monitoring device according to claim 7, wherein the piezoelectric actuator comprises:
a suspension plate being a square suspension plate and permitted to undergo a bending vibration;
an outer frame arranged around the suspension plate;
at least one bracket connected between the suspension plate and the outer frame for elastically supporting the suspension plate; and
a piezoelectric element, wherein a length of a side of the piezoelectric element is smaller than or equal to a length of a side of the suspension plate, and the piezoelectric element is attached on a surface of the suspension plate to drive the suspension plate to undergo the bending vibration in response to an applied voltage.

9. The health monitoring device according to claim 8, wherein the suspension plate has a bulge, a first surface attached on the piezoelectric element and a second surface opposite to the first surface, and the bulge is disposed on the second surface.

10. The health monitoring device according to claim 9, wherein the bulge is formed by an etching process and is a convex structure integrally formed on the second surface.

11. The health monitoring device according to claim 7, wherein the micro pump further comprises a first insulation plate, a conducting plate and a second insulation plate, and the gas inlet plate, the resonance plate, the piezoelectric actuator, the first insulation plate, the conducting plate and the second insulation plate are stacked sequentially.

12. The health monitoring device according to claim 7, wherein the piezoelectric actuator comprises:
a suspension plate being a square suspension plate and permitted to undergo a bending vibration;
an outer frame arranged around the suspension plate;
at least one bracket connected between the suspension plate and the outer frame for elastically supporting the suspension plate, wherein a non-coplanar structure is formed on a surface of the suspension plates and a surface of the outer frame, and a chamber space is maintained between the surface of the suspension plate and the resonance plate; and
a piezoelectric element, wherein a length of a side of the piezoelectric element is smaller than or equal to a length of a side of the suspension plate, and the piezoelectric element is attached on the surface of the suspension plate to drive the suspension plate to undergo the bending vibration in response to an applied voltage.

13. The health monitoring device according to claim 4, wherein the micro pump is a micro pump of a micro-electromechanical system.

14. The health monitoring device according to claim 4, wherein the elastic air bag is in fluid communication with the gas-collecting slot of the gas-collecting actuator through a gas connection channel, and the elastic air bag is allowed to be inflated by the gas transported by the gas-collecting actuator and elastically protrude out of the wearable component.

15. The health monitoring device according to claim 4, wherein an embedding seat is disposed inside the main body, the gas-collecting actuator is carried and positioned in the embedding seat, a gas-collecting slot opening and a communication channel are disposed on the bottom of the embedding seat and are in fluid communication with the gas-collecting slot of the gas-collecting actuator, the communication channel disposed on the bottom of the embedding seat is in fluid communication with the elastic air bag, and the elastic air bag is allowed to be inflated by the gas transported by the gas-collecting actuator and elastically protrude out of the wearable component.

16. The health monitoring device according to claim 4, wherein the micro pump is a micro box pump comprising:
a nozzle plate comprising a plurality of connecting elements, a suspension board and a central aperture, wherein the suspension board is permitted to bend and vibrate, the plurality of connecting elements are adjacent to and connected to edges of the suspension board, the central aperture is formed in a center of the suspension board, the suspension board is securely disposed by the plurality of connecting elements, the plurality of connecting elements elastically support the suspension board, an airflow chamber is formed on the bottom of the nozzle plate, and at least one vacant space is formed among the plurality of connecting elements and the suspension board;
a chamber frame carried and stacked on the suspension board;
an actuating element carried and stacked on the chamber frame, wherein the actuation element is configured to bend and vibrate in a reciprocating manner by an applied voltage;
an insulation frame carried and stacked on the actuating element; and
a conducting frame carried and stacked on the insulation frame,
wherein a resonance chamber is formed among the actuating element, the chamber frame and the suspension board collaboratively, wherein when the voltage is applied to the actuating element, the actuating element drives the nozzle plate to vibrate in resonance, the suspension board of the nozzle plate is driven to vibrate in the reciprocating manner, so as to make the gas flow through the at least one vacant space into the airflow chamber and discharged through the monitoring channel to achieve air transportation.

17. The health monitoring device according to claim 16, wherein the actuating element comprises:
a piezoelectric carrying plate carried and stacked on the chamber frame;
an adjusting resonance plate carried and stacked on the piezoelectric carrying plate; and
a piezoelectric plate carried and stacked on the adjusting resonance plate, wherein the piezoelectric plate is configured to drive the piezoelectric carrying plate and the adjusting resonance plate to bend and vibrate in the reciprocating manner by the applied voltage.
